(19) United States
(12) Patent Application Publication
(10) Pub. No.: US 2007/0043323 A1
(43) Pub. Date: Feb. 22, 2007

(54) Surgical template

(76) Inventor: Christopher Davey, Boston, MA (US)

Correspondence Address: Kevin M. Farrell;Pierce Atwood, Suite 350, One New Hampshire Avenue, Portsmouth, NH, 03801, US

(21) Appl. No.: 11/197,417

(22) Filed: Aug. 4, 2005

(51) Int. Cl. A61M 5/00

(52) U.S. Cl. 604/116

(57) ABSTRACT

The present invention is a surgical template adapted for use in implanting tissue ingrowth cuff assemblies and other semi-permanent or permanent transcutaneous medical devices. The surgical template of the present invention generally includes a body portion and a template portion, wherein the body portion is substantially planar in shape and the template portion protrudes there from. The template portion preferably defines and annular shape and a cavity therein through which a cutting tool can pass in order to extract an appropriate amount of the patient's dermis and connective tissue. The remaining orifice or passageway is sufficient in size to accept a transcutaneous medical device, particularly of the CVC-type described above. The present invention also includes marking means or a handle for properly spacing the catheter or device passageway from the initial incision through which the surgical template is placed.

### BACKGROUND OF THE PRESENT INVENTION

1. FIELD OF INVENTION

The present invention relates generally to surgical tools and instruments, and more specifically to surgical tools and instruments for use in specific surgical procedures requiring precision placement of incisions in a patient's body.

2. HISTORY OF THE RELATED ART

It is often necessary in medically treating a patient to establish long term vascular access to a specific desired interior body site for purposes of administering liquid therapeutic agents, for removing bodily fluids for testing or monitoring, for treatment of bodily fluids before being returned to the body, or for disposal of bodily fluids. In another increasingly common medical procedure, it is desired to deliver a contained heat exchange fluid at a temperature above or below normal body temperature to a specific interior body site for providing localized or general heating or cooling. In still other common medical procedures, such as angioplasty and laparoscopy, medical instrumentation is guided through a pre-positioned catheter line to a particular internal body location to monitor body conditions and/or to perform medical/surgical procedures.

Particularly in the case of administering fluids to, or removing fluids from, the body continuously or periodically over an extended time period, it is known in the medical arts to use what are known as “permanent” catheterization techniques employing subcutaneous-implanted devices such as tunneled central venous catheters (CVCs) for durations ranging from a few weeks to years. Examples of such subcutaneous-implanted and related medical devices are found in U.S. Pat. No. 4,266,999 (Baier); U.S. Pat. No. 4,405,305 (Stephen et al.); U.S. Pat. No. 4,488,877 (Klein et al.); U.S. Pat. No. 4,668,222 (Poirier); U.S. Pat. No. 4,897,081 (Poirier et al.); U.S. Pat. No. 4,935,004 (Cruz); U.S. Pat. No. 5,098,397 (Svensson et al.); U.S. Pat. No. 5,100,392 (Orth et al.); U.S. Pat. No. 5,242,415 (Kantrowitz et al.); U.S. Pat. No. 5,662,616 (Bousquet); U.S. Pat. No. 5,823,994 (Sharkey et al.); U.S. Pat. No. 5,830,184 (Basta); U.S. Pat. No. 5,848,987 (Baudino et al.); U.S. Pat. No. 5,882,341 (Bousquet); U.S. Pat. No. 5,989,213 (Maginot); and U.S. Pat. No. 6,033,382 (Basta), each of which is incorporated herein by reference. Examples of therapeutic regimens requiring such long-term continuous or periodic access to a specific internal body location include parenteral feeding, chemotherapy, antibiotic administration, dialysis, and others.

Generally, the length of time the patient will be catheterized dictates whether a physician will utilize a “temporary” catheterization technique or a “permanent” catheterization technique For example, a procedure in which a clot is aspirated from a blood vessel typically includes placing the catheter in the blood vessel for a relatively short period of time such as a few minutes to a few hours and then withdrawing the catheter once the clot has been removed. Therefore, when performing such an aspiration procedure, it is common for a physician to use the temporary catheterization technique to place the catheter in the blood vessel of the patient.

On the other hand, when a procedure is performed to effect hemodialysis, a physician may place a catheter in the blood vessel for a relatively long period of time. In particular, a patient suffering from kidney failure who is involved in a hemodialysis regimen typically requires a dialysis session three times per week for an indefinite period of time whereby extra fluid, chemicals, and wastes are removed from her body. A patient who is involved in such a hemodialysis regimen may need a catheter placed in her blood vessel for a relatively long period of time in order to provide a ready means for vascular access into his/her bloodstream over such relatively long period of time.

Permanent catheterization techniques typically entail inserting a “permanent” catheter into a patient's blood vessel using a “tunneled catheter technique.” Although done regularly, current CVC designs seriously compromise the skin's ability to protect the body from infection. CVC-related infection is a serious health problem that significantly increases the morbidity rate and cost of catheter usage. All previous attempts to modify tunneled CVC designs to reduce infection have failed to significantly decrease this cost or the morbidity rate. The primary reason for the failure of conventional CVCs is that none of the modified versions effectively block the path of microorganisms through the skin and into the body.

Unlike peritoneal catheters, tunneled CVCs need to have their tips placed in a very specific location, typically the Superior Vena Cava/Right Atrial Junction (SVC/RA), in order to function properly over many months. Interventional radiologists are acutely aware of the need for highly precise tip placement because they are most frequently called on to resolve CVC complications. With currently available technology, the ability to precisely position CVC tips in the SVC/RA is enabled largely by the freedom to adjust/position the cuff location anywhere within the subcutaneous tunnel length.

It has now been found, however, that a CVC apparatus which includes an adjustable epidermal tissue ingrowth cuff assembly according to the present invention overcomes these problems and deficiencies of the prior art CVC devices. For example, the apparatus and methods disclosed in U.S. Patent Application No. 2004/0236314 to Mark A. Saab, incorporated herein by reference, allow a physician to place a fixed epidermal tissue ingrowth cuff assembly within a skin incision site and, subsequently, to adjust the location of the distal (internal) tip of a catheter assembly associated with the tissue ingrowth cuff assembly to precisely position the catheter tip at the desired body site without disturbing, moving, or stressing the fixed tissue ingrowth cuff.

Eliminating the path microbes use to enter the body is the key to reducing CVC cost and morbidity associated with infection. Developing technology provides for a device having a tissue ingrowth cuff assembly that can be placed at a fixed location at the incision site in combination with a catheter component which can be subcutaneously attached to the cuff assembly in such a way that its length and tip position is not constrained by the choice of cuff location. Careful manufacturing processes hermetically seal all exterior device surfaces that will reside above the skin. All surfaces that require assembly during device placement are designed to reside under the skin. The novel assembly of current tissue ingrowth cuff assemblies eliminates the principal and most common path for microbial migration from the skin surface to the interior of the patient via the percutaneous entry site.

Nevertheless, while developments advanced tissue ingrowth cuff assemblies have resulted in numerous improvements related to patient care and health, the procedures and mechanisms for placing any ancillary medical devices into a patient necessarily require precision and care. As such, there is a need in the art for a surgical tool or template that will assist a physician or surgeon in the placement of any medical devices such as tissue ingrowth cuff assemblies. Moreover, there is a need in the art for a surgical tool or template that will improve the efficiency of ingrowth cuff assemblies and minimize the amount of trauma and damage done to the patient's dermis and connective tissue. Lastly, there is a need in the art for such a surgical tool or template that can be readily manufactured, stored in sterile conditions, easily used and effectively cleaned or disposed of following use.

### SUMMARY OF THE PRESENT INVENTION

Accordingly, the present invention includes a surgical template adapted for use in implanting tissue ingrowth cuff assemblies and other semi-permanent or permanent transcutaneous medical devices. The surgical template of the present invention generally includes a body portion and a template portion, wherein the body portion is substantially planar in shape and the template portion protrudes there from. In preferred embodiments, the template portion defines and annular shape and a cavity therein through which a cutting tool can pass in order to extract an appropriate amount of the patient's dermis and connective tissue. A base portion defining part of the cavity prevents the cutting tool from penetrating any of the patient's non-superficial tissues or organs. The remaining orifice or passageway is sufficient in size to accept a transcutaneous medical device, particularly of the CVC-type described above. The present invention also includes marking means for properly spacing the catheter or device passageway from the initial incision through which the surgical template is placed.

In an alternate embodiment described below, the surgical template includes a handle affixed to the trailing edge of the body portion as well as a leading edge that is beveled. These features together allow a surgeon to use the present invention both as a template and also as a cutting tool for performing the gross dissection. Once the initial incision is made, the surgical template is inserted beneath the patient's dermis and it can be used to separate the dermis and connective tissue from the less superficial anatomy, thereby forming a pocket suitable for receiving a transcutaneous medical device.

In its preferred embodiments discussed below, the present invention is composed of a material that can be sterilized and readily disposed after use. Moreover, as the surgical template of the present invention can be mass-produced, the relative sizes, shapes and depths of its features can be varied in order to suit different patients with differing needs. As such, the present invention provides assistance to physicians and surgeons implanting transcutaneous medical devices. Moreover, the present invention in its various embodiments will reduce the amount of trauma inflicted upon the patient during implantation and reduce the risk of CVC-based infections. These and various other features and benefits of the present invention are described more fully below with reference to the following drawings.

### BRIEF DESCRIPTION OF THE DRAWINGS

FIG. 1 is a perspective view of a surgical template in accordance with the present invention.

FIG. 2 is a plan view of the surgical template of the present invention.

FIG. 3 is a cross-sectional view of the surgical template of the present invention.

FIG. 4 is a partial view of the surgical template of the present invention in use with a typical patient according to a typical catheterization procedure.

FIG. 5 is a perspective view of a surgical template in accordance with an alternate embodiment of the present invention.

FIG. 6 is a plan view of the alternate embodiment of the surgical template of the present invention.

FIG. 7 is a cross-sectional view of the alternative embodiment of the surgical template of the present invention.

### DETAILED DESCRIPTION OF THE PREFERRED EMBODIMENTS

The present invention is an improved surgical template 10 for use in various surgical procedures, including for example catheterization procedures. In particular, the present invention provides a novel tool or template for use by surgeons in determining the proper placement of catheterization ports, including the placement of the initial incision and the creation of the passage through which the catheter is directed. Moreover, the surgical template 10 of the present invention accomplishes these goals while securing the health of the patient and minimizing the discomfort and pain associated with the placement of the catheterization port.

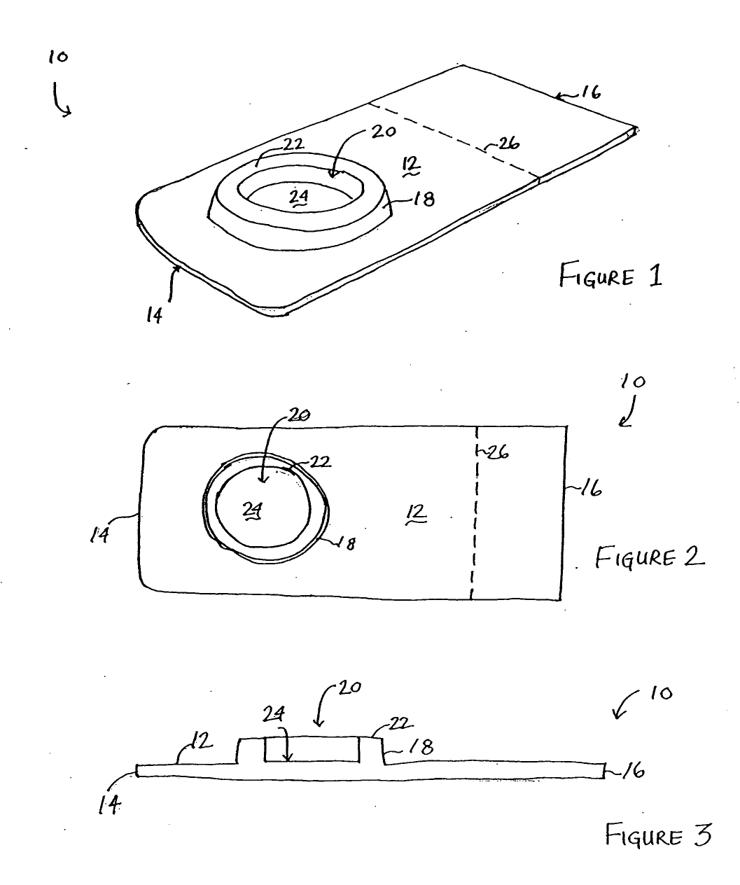

FIG. 1 is a perspective view of the surgical template 10 in accordance with the present invention. The surgical template 10 includes a body portion 12 defining a leading edge 14 and a trailing edge 16, wherein the leading edge 14 and the trailing edge 16 are opposed to one another about the body portion 12. The body portion 12 is substantially planar in shape, and preferably composed of a durable, hygienic and material.

A template portion 18 is disposed on the body portion 12 such that is rises from the body portion 12 providing a detectable rise from the surface of the body portion 12. In preferred embodiments, the template portion 18 is annular or ring-shaped, thus defining a circular cavity 20 therein and having a circular rim 22 about its perimeter. As the outer surfaces of both catheters and tissue ingrowth cuff assembly are generally cylindrical in shape, the template portion 18 is preferably annular as well. However, for applications in which the tissue ingrowth cuff assembly, for example, is not cylindrical in shape the shape of the template portion 18 would be redesigned accordingly. The cavity 20 is formed by the junction between the template portion 18 and the body portion 12, at which junction a base portion 24 is formed for prohibiting penetration of any cutting instruments through the cavity 20 itself.

As shown in FIG. 2, the present invention also includes a marking means 26 that is included in the surgical template 10 of the present invention. The marking means 26 is disposed at a predetermined distance from the template portion 18, wherein the predetermined distance corresponds to a desirable separation between the initial incision and the placement of the ingrowth cuff apparatus. For the foregoing reasons, the marking means 26 are located a predetermined distance from the template portion 18, for example between 2 and 5 centimeters from the center of the cavity 20. The marking means 26 itself can be provided through any number of means, either visual or tactile, that permit the surgeon performing the procedure to readily appreciate the proper placement of the template portion.

FIG. 3 is a cross-sectional view of the surgical template 10 illustrating in particular features of the cavity 20. The depth of the cavity 20 is defined in part by the relative positions of the body portion 12 and the base portion 24 with respect to the template portion 18. For example, if the body portion 12 is relatively higher up the template portion 18 as compared to the base portion 24, then the cavity 20 will be relatively deep, thus permitting greater penetration of any cutting instruments. Conversely, if the body portion 12 is relatively lower down the template portion 18 as compared to the base portion 24, then the cavity 20 will be relatively shallow, thus prohibiting greater penetration of any cutting instruments. It is contemplated that the present invention will be constructed across a range of relative cavity 60 depths such that a surgeon can select the proper surgical template 40 size depending on the character and thickness of the patient's dermis.

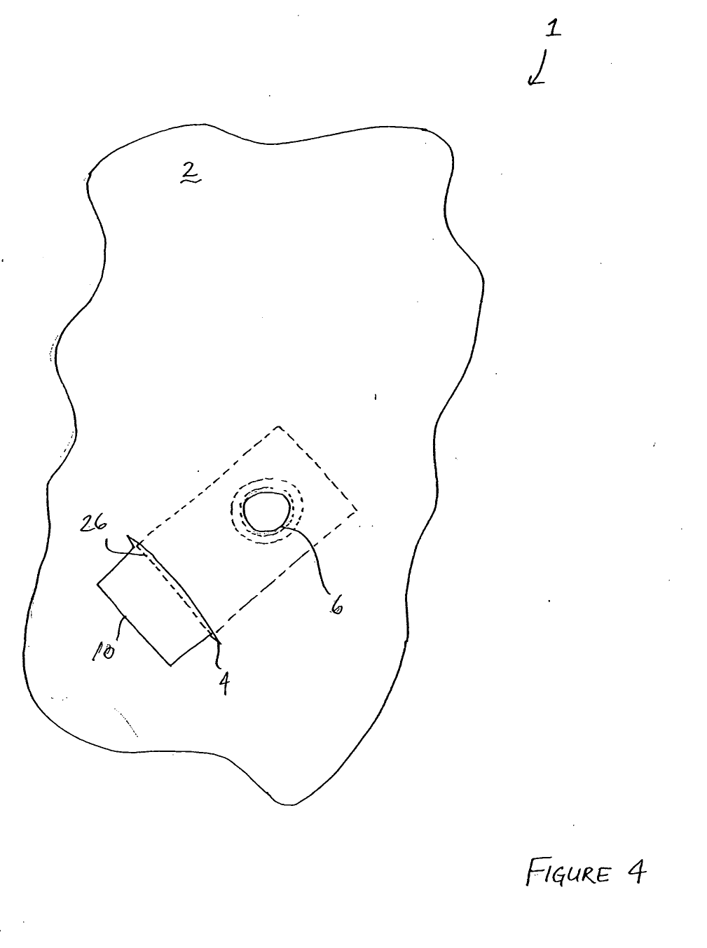

Use of the present invention is illustrated in FIG. 4 on a typical patient 1 having a dermis 2 and underlying connective tissue (not shown). As previously noted, in placing a tissue ingrowth cuff within the body a surgeon must make two incisions. A first incision 4 provides an opening through which the subcutaneous portion of the tissue ingrowth cuff is placed, and a second incision 6 is made for passing the catheter through the dermis and into the subcutaneous portion of the tissue ingrowth cuff. Accordingly, it is customary and preferable for a surgeon to separate these incisions such that any healing, infection or ingrowth occurring at one incision will not affect the other. More particularly, it is desirable to separate the first incision 4 from the second incision 6 such that any device implanted beneath the skin will not tear, stress or otherwise traumatize the wound formed by the first incision 4.

As the first incision 4 is generally linear in form, the surgeon can insert the surgical template 10 beneath the dermis of the patient until the marking means 26 is aligned with the first incision 4. Then, the surgeon can make the second incision 6 and remove the dermis 2 within the cavity 20 without any fear of coring too deep into the patient's body due to the presence of the base portion 24. Once the dermis over the cavity 20 has been removed, the surgeon can withdraw with surgical template 10 and begin placement of the tissue ingrowth cuff through the first incision 4.

In addition to passing through the dermis 2, access to the internal physiology of the patient 1 also typically involves penetrating layers of fatty and connective tissues. For example, when the surgeon is inserting the surgical template 10 of the present invention within the first incision 4, he or she must typically lift or otherwise separate the dermis 2 from the underlying connective tissue. This procedure is known as blunt or gross dissection, and it typically involves the use of forceps or other non-bladed instruments for roughly separating the dermis 2 from the underlying fatty and connective tissues. Although blunt dissection is sufficiently useful for the task at hand, it remains and inefficient and overly traumatic procedure and may inadvertently increase the incidence of infection in placing the catheter. In particular, the pulling and tugging actions of blunt dissection may create more tissue damage that will inevitably lead to the possibility of infection, and in close proximity to the location of the catheter itself as well.

In order to decrease the trauma inflicted through the placement of the tissue ingrowth cuff, an alternate embodiment of the present invention is presented herein.

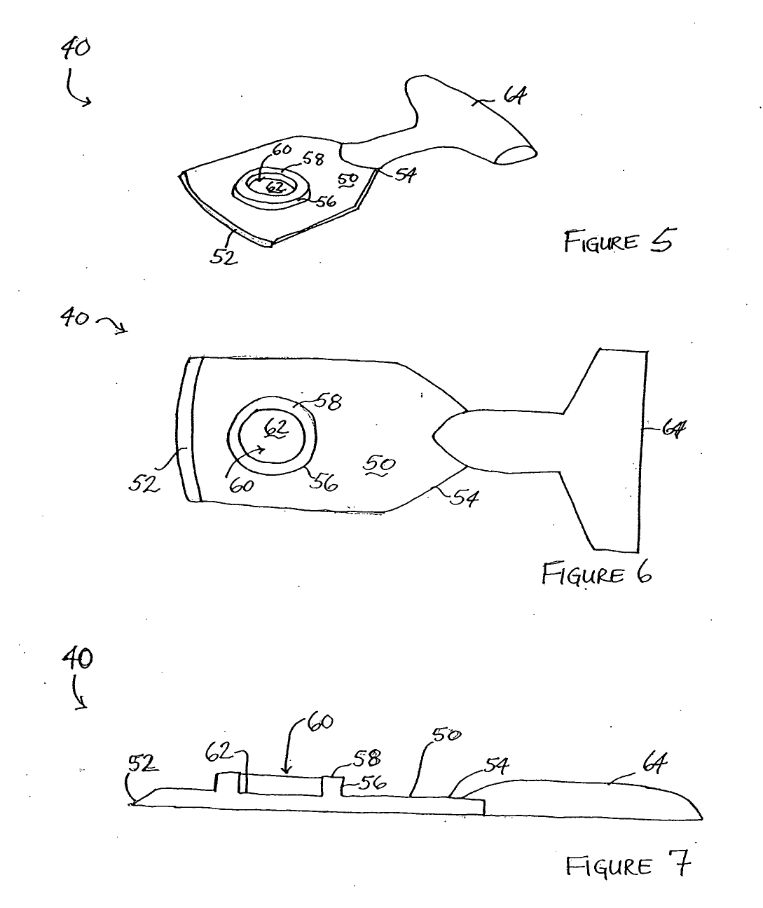

FIG. 5 is a perspective view of a surgical template 40 in accordance with an alternate embodiment of the present invention. The surgical template 40 includes a body portion 50 that is bounded by a leading edge 52 and a trailing edge 54. The body portion 50 is preferably substantially planar in order to facilitate entry under the patient's dermis. The leading edge 52 is beveled or bladed as discussed further below, and the trailing edge is affixed to or otherwise in communication with a handle 64. In its preferred embodiments, the surgical template 40 is of a durable and hygienic material.

A template portion 56 is disposed on the body portion 50 such that is rises from the body portion 50 providing a detectable rise from the surface of the body portion 50. In preferred embodiments, the template portion 56 is annular or ring-shaped, thus defining a circular cavity 60 therein and having a circular rim 58 about its perimeter. As the outer surfaces of both catheters and tissue ingrowth cuff assembly are generally cylindrical in shape, the template portion 56 is preferably annular as well. However, for applications in which the tissue ingrowth cuff assembly, for example, is not cylindrical in shape the shape of the template portion 56 would be redesigned accordingly.

Additional features of the surgical template 40 are shown in FIGS. 6 and 7, which are plan and cross-sectional views, respectively, of the present invention. As shown in both FIGS. 6 and 7, the leading edge 52 is preferably beveled or bladed in order to provide a cutting or separating edge for performing the gross dissection discussed above. The surgical template 40, including the leading edge 52, is preferably comprised of a sterile material that can be disposed of following the procedures described herein.

The surgical template 40 also includes a handle 64 that a surgeon can utilize to guide and direct the surgical template 40 during the gross dissection and placement phases of the procedure described above. The handle 64 is disposed at the trailing edge 54 of the body portion 50 such that the presence of the handle 64 will alert the surgeon as to the position of the template portion 56 relative to the first incision. Functionally, the handle 64 performs the same function as the marking means 26 discussed above with reference to the previous embodiment, i.e. it notifies the surgeon that the second incision through the dermis can be made. However, the surgical template 40 may also include a marking means (not shown) in addition to the handle 64 to ensure proper placement of the template portion 56 under the patient's dermis. In its preferred embodiments, the handle 64 and/or supplemental marking means are disposed approximately 2 to 5 centimeters from the center of a cavity 60.

In further embodiments, the surgical template 40 may include a leading edge 52 that is serrated or scalloped blade as opposed to a straight edged or beveled blade. Moreover, the surgical template 40 may include additional cutting edges disposed on sides of the body portion 50 adjacent to the handle 64 such that a surgeon can perform the blunt dissection procedure in a swinging or slicing motion as opposed to a pushing or scraping motion. The exact configuration of the surgical template 40 including the cutting edges and type of edges used is considered a design and ergonomic concern, and should not be understood to be limiting the scope of the present invention.

As in the prior embodiment, the surgical template 40 includes the cavity 60 that is defined in part by a base portion 62. Also noted in the prior embodiment, the relative positions of the base portion 62 and the body portion 50 relative to the template portion 56 define the depth or shallowness of the cavity 60. It is contemplated that the present invention will be constructed across a range of relative cavity 60 depths such that a surgeon can select the proper surgical template 40 depending on the character and thickness of the patient's dermis.

In each of the foregoing embodiments, the surgical template is preferably composed of a sterile and hygienic material that can be easily produced and readily disposed following use. In particular, the present invention may be formed from stainless steel, titanium, aluminum alloy, conventional plastic or nylon, composite materials such as graphite or carbon fiber, and any other synthetic or metallic material known in the art of surgical instruments. Likewise, the present invention may be comprised of mixtures of the above materials. For example, the body portion and template portion may be of one material selected from the group above while the handle is formed from a different material. As with regards to the blade-type and cutting edges outlined above, the consideration of materials for the present invention should be understood as an economic and design choice primarily, as the present invention is readily adapted to be implemented in a number of materials.

As noted above, the present invention provides a useful and novel solution to the problems associated with the implantation of transcutaneous medical devices. Moreover, the surgical template of the present invention can be readily designed and constructed so as to be a cost-effective and disposable feature of CVC-type procedures. Additionally, the features of the present invention will aid in decreasing infections borne from transcutaneous medical devices, which will in turn improve the overall health and well being of the patient.

While the present invention has been described above with reference to its preferred embodiments, it should be understood that various permutations of these embodiments can be readily devised by those skilled in the art without departing from the scope of the present invention as defined in the following claims.

I claim:

1. A surgical template comprising:
a body portion defining a substantially planar surface, a leading edge and a trailing edge; and
a template portion disposed on the body portion between the leading edge and the trailing edge, the template portion cooperating with the planar surface to define a cavity through which a surgical instrument may partially pass.

2. The surgical template of claim 1 wherein the template portion is annular.

3. The surgical template of claim 1 further comprising marking means disposed between the template portion and the trailing edge for aligning the surgical template.

4. The surgical template of claim 3 wherein the marking means and the template portion are disposed at a predefined distance corresponding to a distance between an incision and an entry point for catheterization.

5. The surgical template of claim 1 wherein the leading edge defines a blade for gross dissection during surgical procedures.

6. The surgical template of claim 1 further comprising a handle disposed at the trailing edge.

7. The surgical template of claim 1 further comprising a handle disposed at the trailing edge and further wherein the leading edge defines a blade for gross dissection during surgical procedures.

8. The surgical template of claim 1 further comprising a handle disposed at the trailing edge and marking means disposed between the handle and the template portion.

9. The surgical template of claim 1 wherein the body portion is comprised of a metallic material.

10. The surgical template of claim 1 wherein the template portion is comprised of a metallic material.

11. The surgical template of claim 1 further comprising a base portion disposed within the cavity defined by the template portion, the base portion being substantially coplanar with the body portion.

12. The surgical template of claim 3 wherein the marking means is disposed a predetermined distance from the center of the template portion.

13. The surgical template of claim 12 wherein the predetermined distance is between 2 and 5 centimeters.

14. The surgical template of claim 5 wherein the blade is a straight edge.

15. The surgical template of claim 5 wherein the blade is a serrated edge.
